(19) United States
(12) Patent Application Publication
(10) Pub. No.: US 2025/0121142 A1
(43) Pub. Date: Apr. 17, 2025

(54) RETRACTION MECHANISM FOR SAFE INJECTION SYSTEM

(71) Applicant: CREDENCE MEDSYSTEMS, INC., Menlo Park, CA (US)

(72) Inventors: Mina M. Leung, Mountain View, CA (US); Stephen H. Diaz, Palo Alto, CA (US); Jeff Tillack, Wake Forest, NC (US); Alan E. Shluzas, San Carlos, CA (US)

(73) Assignee: CREDENCE MEDSYSTEMS, INC., Menlo Park, CA (US)

(21) Appl. No.: 18/911,855

(22) Filed: Oct. 10, 2024

Related U.S. Application Data
(60) Provisional application No. 63/589,708, filed on Oct. 12, 2023.

(51) Int. Cl. A61M 5/34

(52) U.S. Cl. CPC A61M 5/343

(57) ABSTRACT

A system for safe injections includes a syringe body having proximal and distal ends, a syringe interior, and a syringe flange at the proximal end thereof. The system also includes a needle hub assembly coupled to the distal end of the syringe body. The needle hub assembly includes a needle hub coupled to the distal end of the syringe body, and a needle removably coupled to the needle hub. The system further includes a stopper member disposed in the syringe interior. Moreover, the system includes a plunger member coupled to the stopper member. The plunger member includes a plunger housing defining a plunger interior, a spring and a latch, and a needle catch and a trigger. The spring and the latch are integrally formed, and the needle catch and the trigger are integrally formed.

The present application claims priority to U.S. Provisional Patent Application Ser. No. 63/589,708, filed on Oct. 12, 2023 under attorney docket number CM.30030.03 and entitled “RETRACTION MECHANISM FOR SAFE INJECTION SYSTEM.” This application includes subject matter similar to the subject matter described in the following co-owned U.S. patent applications: (1) U.S. Utility patent application Ser. No. 14/696,342, filed Apr. 24, 2015 under attorney docket number CM.20003.00, and entitled “SYSTEM AND METHOD FOR SAFETY SYRINGE”; (2) U.S. Utility patent application Ser. No. 14/543,787, filed Nov. 17, 2014 under attorney docket number CM.20002.00, and entitled “SYSTEM AND METHOD FOR DRUG DELIVERY WITH A SAFETY SYRINGE”; (3) U.S. Utility patent application Ser. No. 14/321,706, filed Jul. 1, 2014 under attorney docket number CM.20001.00, and entitled “SAFETY SYRINGE”; (4) U.S. Utility patent application Ser. No. 15/801,239, filed Nov. 1, 2017 under attorney docket number CM.20011.00, and entitled “SYSTEM AND METHOD FOR SAFETY SYRINGE”; (5) U.S. Utility patent application Ser. No. 15/801,259, filed Nov. 1, 2017 under attorney docket number CM.20012.00, and entitled “SYSTEM AND METHOD FOR SAFETY SYRINGE”; (6) U.S. Utility patent application Ser. No. 15/801,281, filed Nov. 1, 2017 under attorney docket number CM.20013.00, and entitled “CARTRIDGE SAFETY INJECTION SYSTEM AND METHODS”; (7) U.S. Utility patent application Ser. No. 15/801,304, filed Nov. 1, 2017 under attorney docket number CM.20015.00, and entitled “SYSTEM AND METHOD FOR SAFETY SYRINGE”; (8) U.S. Utility patent application Ser. No. 16/435,429, filed Jun. 7, 2019 under attorney docket number CM.20019.00, and entitled “SYSTEM AND METHOD FOR SAFETY SYRINGE”; (9) U.S. Utility patent application Ser. No. 16/798,188, filed Feb. 21, 2020 under attorney docket number CM.20023.00, and entitled “SYSTEM AND METHOD FOR SAFETY SYRINGE”; (10) U.S. Utility patent application Ser. No. 16/837,835, filed Apr. 1, 2020 under attorney docket number CM.20025.00, and entitled “POLYMERIC INJECTION SYSTEMS”; (11) U.S. Utility patent application Ser. No. 16/908,531, filed Jun. 22, 2020 under attorney docket number CM.20026.00, and entitled “INJECTION SYSTEM AND METHOD”; (12) U.S. Utility patent application Ser. No. 17/031,108, filed Sep. 24, 2020 under attorney docket number CM.20027.00, and entitled “SYSTEM AND METHOD FOR SAFETY SYRINGE”; and (13) U.S. Provisional Patent Application Ser. No. 63/046,517, filed Jun. 30, 2020 under attorney docket number CM.30028.00, and entitled “SYSTEM AND METHOD FOR SAFETY SYRINGE.” The contents of the above-mentioned applications are fully incorporated herein by reference as though set forth in full.

### FIELD OF THE INVENTION

The present invention relates generally to safe injection systems, devices, and processes, and more particularly to systems, devices, and methods for retracting needles at least partially into injection systems after completing injections in healthcare environments.

### BACKGROUND

Millions of syringes, such as that depicted in FIG. 1A 2, are consumed in healthcare environments every day. A typical syringe 2 includes a tubular body 4, a plunger 6, and an injection needle 8. As shown in FIG. 1B, such a syringe 2 may be utilized not only to inject fluid into a patient, but also to withdraw or expel fluid out of or into a container such as a medicine bottle, vial, bag, or other drug containment system 10. Indeed, due to regulatory constraints in some countries such as the United States as well as sterility maintenance concerns, upon use of a medicine bottle 10 with a syringe 2 as shown in a particular patient's environment, such medicine bottle may only be utilized with a single patient and then must be disposed of-causing significant medical waste from bottle and remaining medicine disposal, and even contributing to periodic shortages of certain critical drugs.

Referring to FIG. 2A, three Luer-type syringes 12 are depicted, each having a Luer fitting geometry 14 disposed distally, so that they may be coupled with other devices having similar mating geometry, such as the Luer manifold assembly 16 depicted in FIG. 2B. The Luer manifold assembly of FIG. 2B may be used to administer liquid drugs to the patient intravenously with or without the use of an intravenous infusion bag. The Luer fittings 14 of the syringes of FIG. 2A may be termed the “male” Luer fittings, while those of FIG. 2B 18 may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting 14 which are configured to engage a flange on the female fitting 18 and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during the loading to provide a Luer coupling. The use of needle injection configurations, on the other hand, carries with it the risk of a sharp needle contacting or poking a person or structure that is not desired. For this reason, so called “safety syringes” have been developed.

One embodiment of a safety syringe 20 is shown in FIG. 3, wherein a tubular shield member 22 is spring biased to cover the needle 8 when released from a locked position relative to the syringe body 4. Another embodiment of a safety syringe 24 is shown in FIGS. 4A-4B. With such a configuration, after full insertion of the plunger 6 relative to the syringe body 4, the retractable needle 26 is configured to retract 28, 26 back to a safe position within the tubular body 4, as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for pre-filled syringe assemblies such as those depicted in FIGS. 5A and 5B, which generally include a syringe body, or “drug enclosure containment delivery system”, 34, a plunger tip, plug, or stopper 36, and a distal seal or cap 35 which may be fitted over a Luer type interface (FIG. 5A shows the cap 35 in place; FIG. 5B has the cap removed to illustrate the Luer interface 14. Liquid medicine may reside in the volume, or medicine reservoir 40 between the distal seal 35 and the distal end 37 of the stopper member 36. The stopper member 36 may include a standard butyl rubber material and may be coated, such as with a biocompatible lubricious coating (e.g., polytetrafluoroethylene (“PTFE”)), to facilitate preferred sealing and relative motion characteristics against the associated syringe body 34 structure and material. The proximal end of the syringe body 34 in FIG. 5B includes a conventional integral syringe flange 38), which is formed integral to the material of the syringe body 34. The flange 38 is configured to extend radially from the syringe body 34 and may be configured to be a full circumference, or a partial circumference around the syringe body 34. A partial flange is known as a “clipped flange” while the other is known as a “full flange.” The flange is used to grasp the syringe with the fingers to provide support for pushing on the plunger to give the injection. The syringe body 34 preferably includes a translucent material such as a glass or polymer. To form a contained volume within the medicine chamber or reservoir 40, and to assist with expulsion of the associated fluid through the needle, a stopper member 36 may be positioned within the syringe body 34. The syringe body may define a substantially cylindrical shape (i.e., so that a plunger tip 36 having a circular cross sectional shape may establish a seal against the syringe body), or be configured to have other cross sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus certain suppliers have moved to more “vertical” solutions, such as that 41 featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Moreover, safe injection systems, especially those configured for use with standard injection system components may have needle retraction systems with sizable numbers of components. Implementing such needle retraction systems (i.e., having sizable numbers of components) increases the complexity, potential for error during assembly and operation, size, and cost of safe injection systems.

There is a need for safe injection systems which address shortcomings of currently-available configurations. In particular, there is a need for safe injection systems that utilize needle retraction systems having a minimum number of components to withdraw needles at least partially into injection systems after completing injections. It is also desirable that such safe injection systems may utilize the existing and relatively well-controlled supply chain of conventionally delivered off-the-shelf components, and the corresponding assembly machinery and personnel.

### SUMMARY

Embodiments are directed to injection systems. In particular, the embodiments are directed to microliter range injection systems that include at least some off-the-shelf syringe components.

In one embodiment, a system for safe injections includes a syringe body having proximal and distal ends, a syringe interior, and a syringe flange at the proximal end thereof. The system also includes a needle hub assembly coupled to the distal end of the syringe body. The needle hub assembly includes a needle hub coupled to the distal end of the syringe body, and a needle removably coupled to the needle hub. The system further includes a stopper member disposed in the syringe interior. Moreover, the system includes a plunger member coupled to the stopper member. The plunger member includes a plunger housing defining a plunger interior, a spring and a latch, and a needle catch and a trigger. The spring and the latch are integrally formed, and the needle catch and the trigger are integrally formed.

In one or more embodiments, the spring is disposed in the plunger interior. The needle catch may be disposed in the plunger interior. The trigger may be disposed in the plunger interior.

In one or more embodiments, the latch is disposed at least partially in the plunger interior. The plunger body may also define a lateral opening therein. The latch may have an extended configuration and a relaxed configuration. When the latch is in the extended configuration, the latch may be configured to extend out of the plunger interior through the lateral opening and to interfere with the plunger body adjacent the lateral opening to limit proximal movement of the latch relative to the plunger body. When the latch is in the extended configuration, the latch may compress the spring.

In one or more embodiments, the spring includes a stack of loops formed from a wire, and the latch includes an orthogonal loop formed from the wire and orthogonal to the stack of loops including the spring. The orthogonal loop may include an opening smaller than a width of the wire. The orthogonal loop may include a straight section.

In one or more embodiments, the needle includes a geometric feature at a proximal end thereof. The needle catch may define a funnel at a distal end thereof, and an opening in the center of the funnel. The funnel may be configured to guide the geometric feature of the needle into the opening and the needle catch. The needle catch may include a needle latch configured to limit distal movement of the geometric feature of the needle relative to the funnel while allowing proximal movement of the geometric feature of the needle relative to the funnel. The needle latch may be self-energizing.

In one or more embodiments, the needle latch includes a needle catch disc disposed in a space defined by the needle catch proximal of the funnel. The needle catch disc may include first and second tabs. The first and second tabs may be pre-bent in a proximal direction. The first tab may be shorter than the second tab. The first tab may be configured to not bend when the geometric feature of the needle moves proximally past the needle catch disc. The second tab may be configured to at least partially elastically bend in a proximal direction to allow the geometric feature of the needle to move proximally past the needle catch disc. The first and second tabs may each be configured to at least partially elastically bend in a proximal direction to allow the geometric feature of the needle to move proximally past the needle catch disc.

In one or more embodiments, the trigger defines a slot configured to receive the latch. The trigger may define an inclined surface configured to move the latch from a relaxed configuration to an extended configuration when the latch moves proximally over the included surface. The trigger and the catch may be configured such that moving the plunger member to position the stopper member at the distal end of the syringe body causes the needle to at partially penetrate the stopper member, engage with the needle catch, and push the trigger proximally relative to the plunger housing and the latch to move the inclined surface proximally away from the latch, and to allow the latch to move from the extended configuration to the relaxed configuration.

In one or more embodiments, the plunger member includes a proximal end cap slidably coupled to a proximal end of the plunger housing. The proximal end cap may define a space configured to minimize transmission of force from the spring upon retraction of the needle at least partially into the plunger interior. The proximal end cap may define a space configured to minimize a sound resulting from retraction of the needle at least partially into the plunger interior.

The aforementioned and other embodiments of the invention are described in the Detailed Description which follows.

### BRIEF DESCRIPTION OF THE DRAWINGS

The foregoing and other aspects of embodiments are described in further detail with reference to the accompanying drawings, in which the same elements in different figures are referred to by common reference numerals, wherein:

FIGS. 1A-5C illustrate various aspects of conventional injection syringe configurations.

FIG. 6 is a side view of a safe injection system according to some embodiments.

FIG. 7 is a cross-sectional and exploded view of a plunger member, showing plunger member components, which form a needle retraction system according to some embodiments.

FIG. 8 is a detailed perspective view of a needle catch/trigger according to some embodiments.

FIG. 9 is a detailed perspective view illustrating assembly of a spring/latch and a needle catch/trigger during assembly of the plunger member according to some embodiments.

FIG. 10 is a mixed perspective and cross-sectional view showing assembly of a needle retraction system according to some embodiments.

FIG. 11 is a detailed top perspective view of a needle retraction system according to some embodiments.

FIG. 12 is a perspective view showing a step in assembly of a plunger member according to some embodiments.

FIGS. 13 and 14 are detailed partial cross-sectional perspective views illustrating assembly of a needle retraction mechanism during assembly of a plunger member according to some embodiments.

FIG. 15 is a detailed cross-sectional view showing a needle retraction mechanism according to some embodiments.

FIGS. 16 and 17 are perspective views illustrating assembly of a plunger member according to some embodiments.

FIGS. 18 to 23 are various cross-sectional views showing use of a safe injection system including a plunger member with a needle retraction system to perform a safe injection according to some embodiments.

FIGS. 24 to 27 are various cross sectional view showing use of a safe injection system including a plunger member with a needle retraction system to perform a safe injection according to some embodiments.

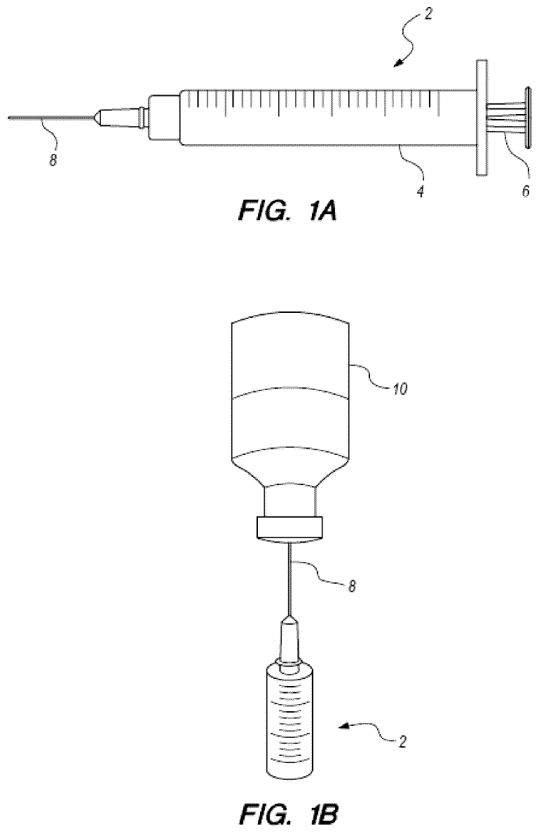

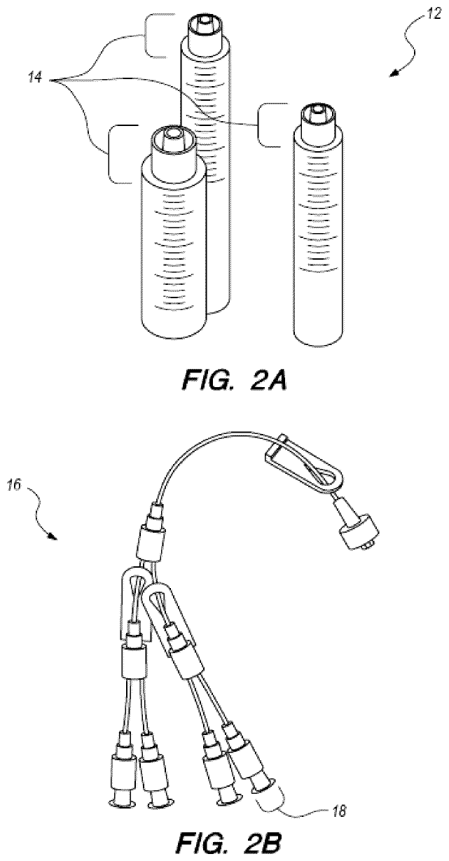

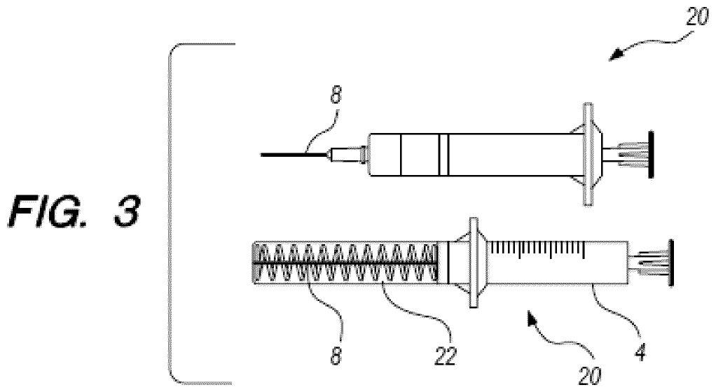

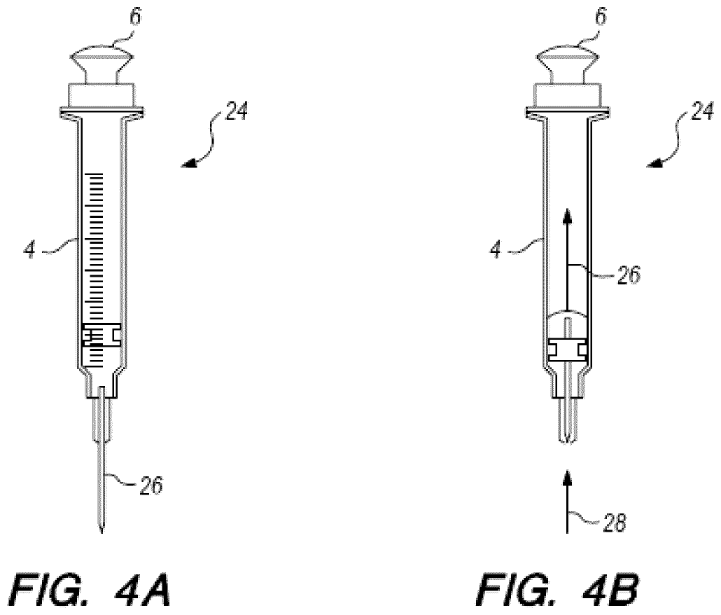

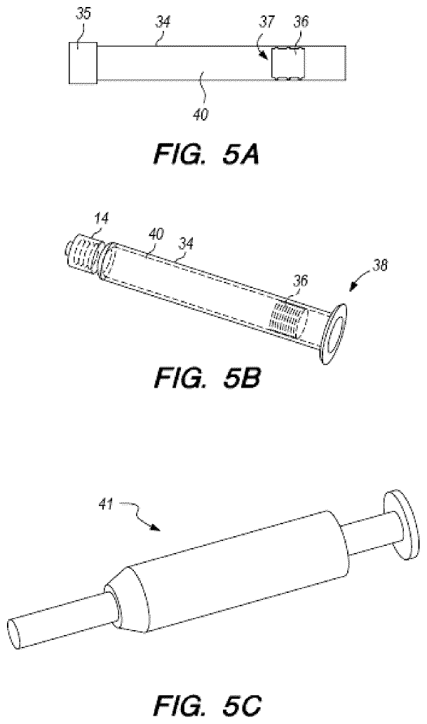

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

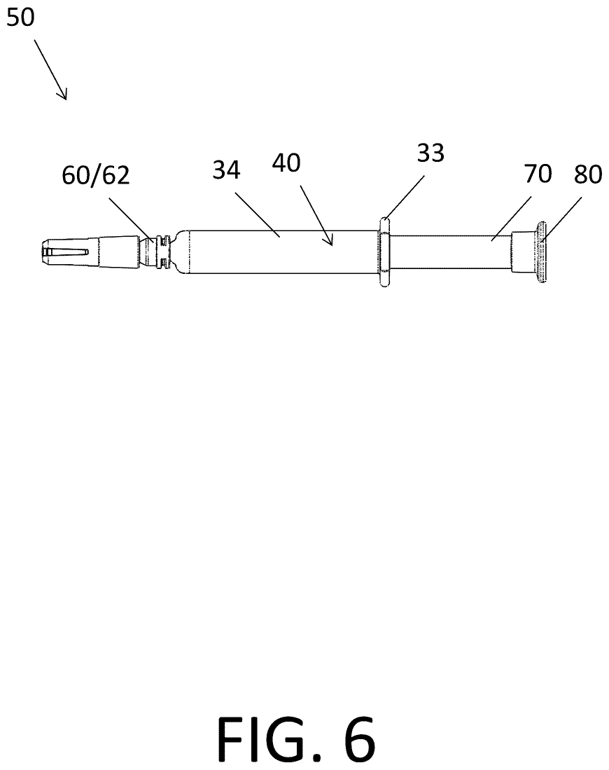

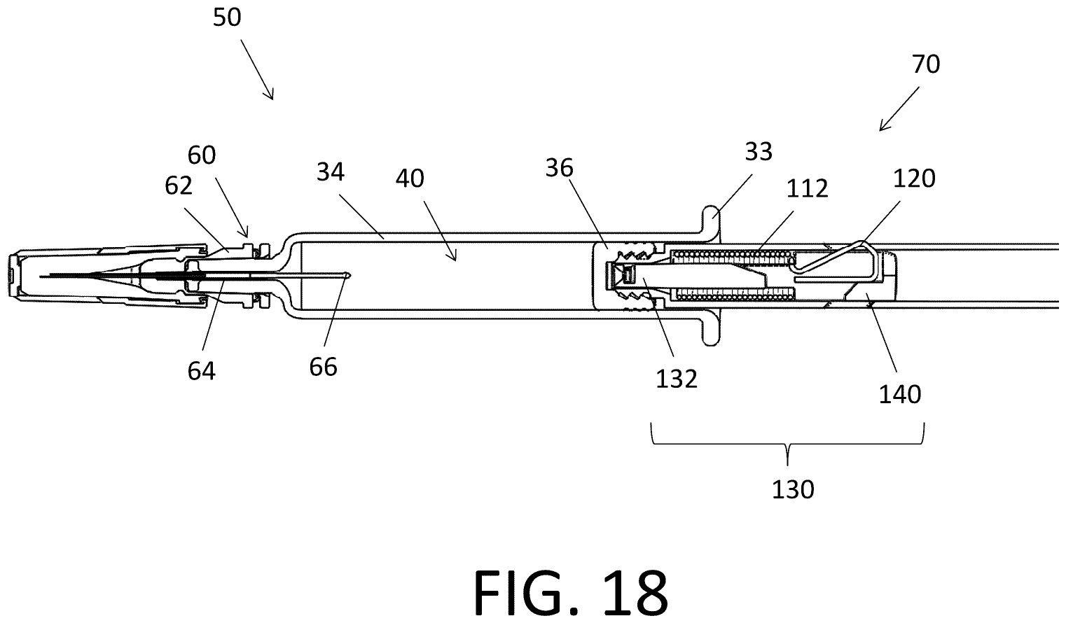

FIGS. 6 to 27 depict a needle retraction system 100 for use with a safe injection system 50 according to some embodiments. As shown in FIGS. 6 and 18, the safe injection system 50 includes a syringe body 34 having proximal and distal ends and defining a syringe interior 40 and a syringe flange 33 at the proximal end thereof. The safe injection system 50 also includes a needle hub assembly 60 including a needle hub 62 coupled to the distal end of the syringe body 34 and a needle 64 removably coupled to the needle hub 62. The safe injection system 50 further includes a stopper member 36 slidably disposed in the syringe interior 40. Moreover, the safe injection system 50 includes a plunger member 70 coupled to the stopper member. Further details regarding the plunger member 70 and plunger member components are described below. Further details regarding other components of the safe injection system 50 are described in U.S. patents application Ser. Nos. 14/696,342, 15/801,239, 15/801,259, 15/801,281, 15/801,304, 16/435,429, 16/837,835, 16/908,531, 17/031,108, and 63/046,517, the contents of which have been incorporated herein by reference.

### Exemplary Needle Retraction System

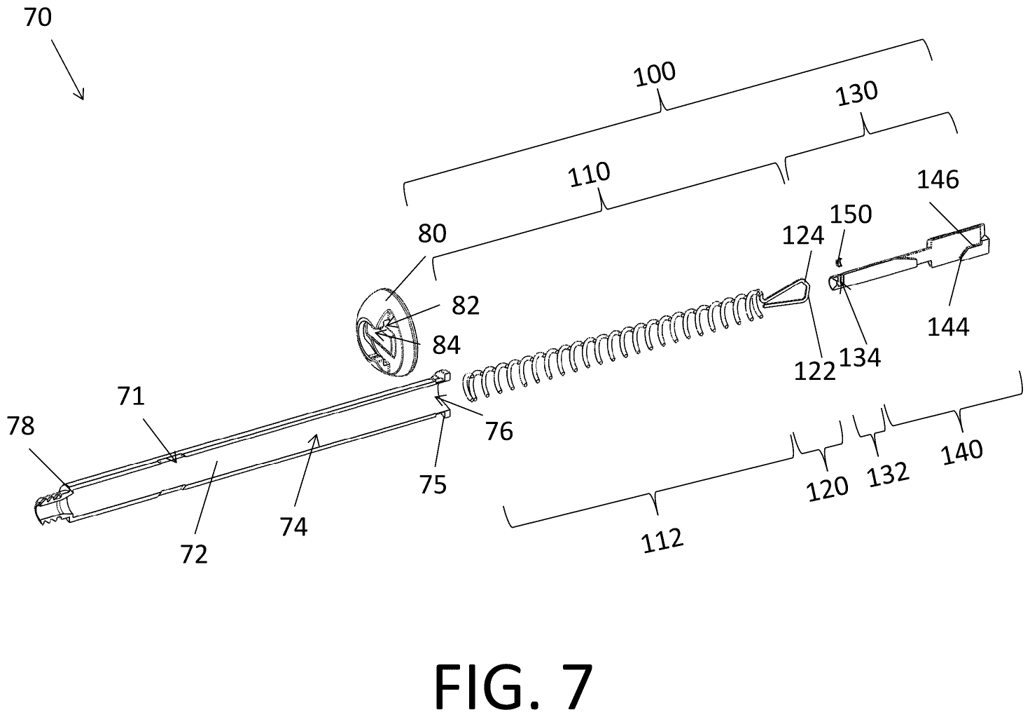

FIG. 7 is a cross-sectional and exploded view of a plunger member 70, showing plunger member components, which form a needle retraction system 100 according to some embodiments. The plunger member 70 includes a plunger housing 72, which defines a plunger interior 74 therein. When not shown in an exploded view, various plunger member components forming the needle retraction system 100 are disposed in the plunger interior 74. The plunger member 70 also includes a proximal end cap 80 configured to seal a proximal end thereof.

The needle retraction system 100 includes an integrally formed spring/latch 110, an integrally formed needle catch/trigger 130, and a needle catch disc 150 disposed in the needle catch/trigger 130. The spring/latch 110 includes a spring portion 112 and a latch portion 120. The spring portion 112 includes a stack of loops (e.g., formed from a wire) and the latch portion 120 includes an orthogonal loop (e.g. also formed from the wire) that is orthogonal to the proximal most loop in the stack of loops. The orthogonal loop includes an opening smaller than a width of the wire to prevent entanglement between the stack of loops and the orthogonal loop. The needle catch/trigger 130 includes a needle catch portion 132 and a trigger portion 140.

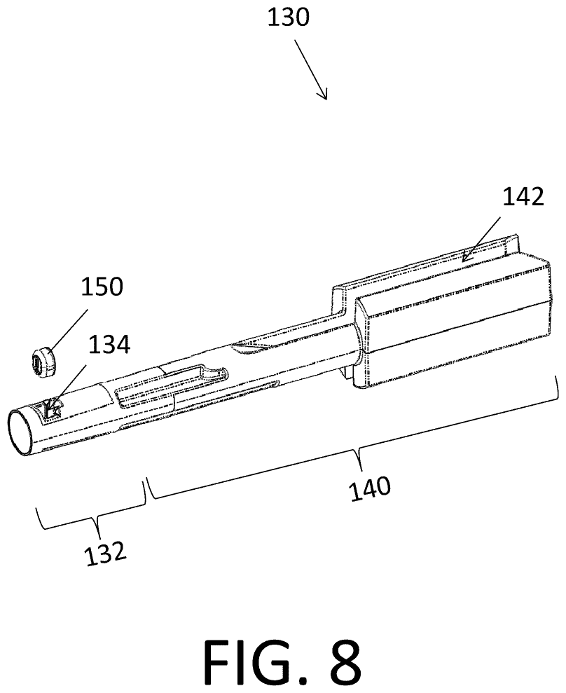

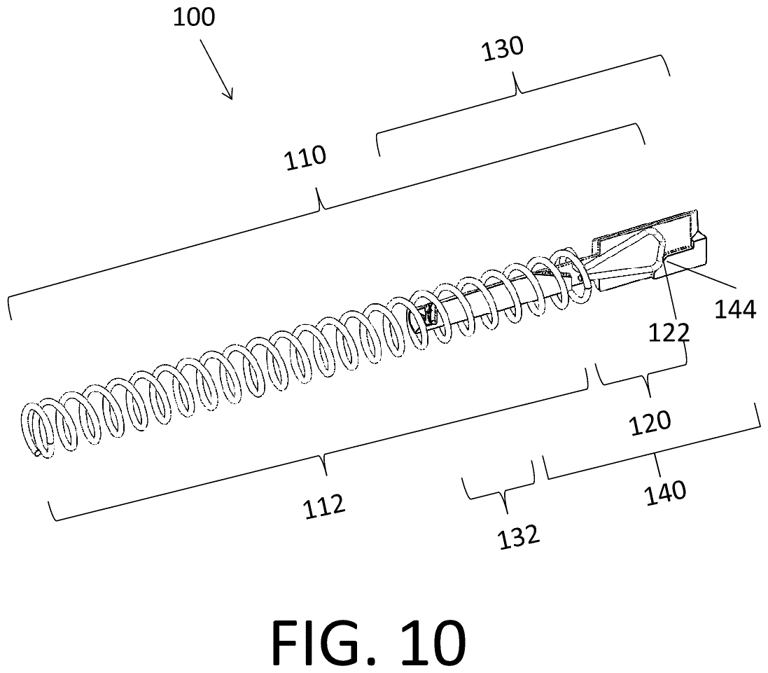

FIG. 8 is a perspective view showing the needle catch/trigger 130 in greater detail, including the needle catch disc 150 and the space 134 defined by the needle catch portion 132 of the needle catch/trigger 130 to receive the needle catch disc 150. The trigger portion 140 of the needle catch/trigger 130 also defines a slot 142 configured to receive a latch portion 120 of the spring/latch 110 (see FIG. 10).

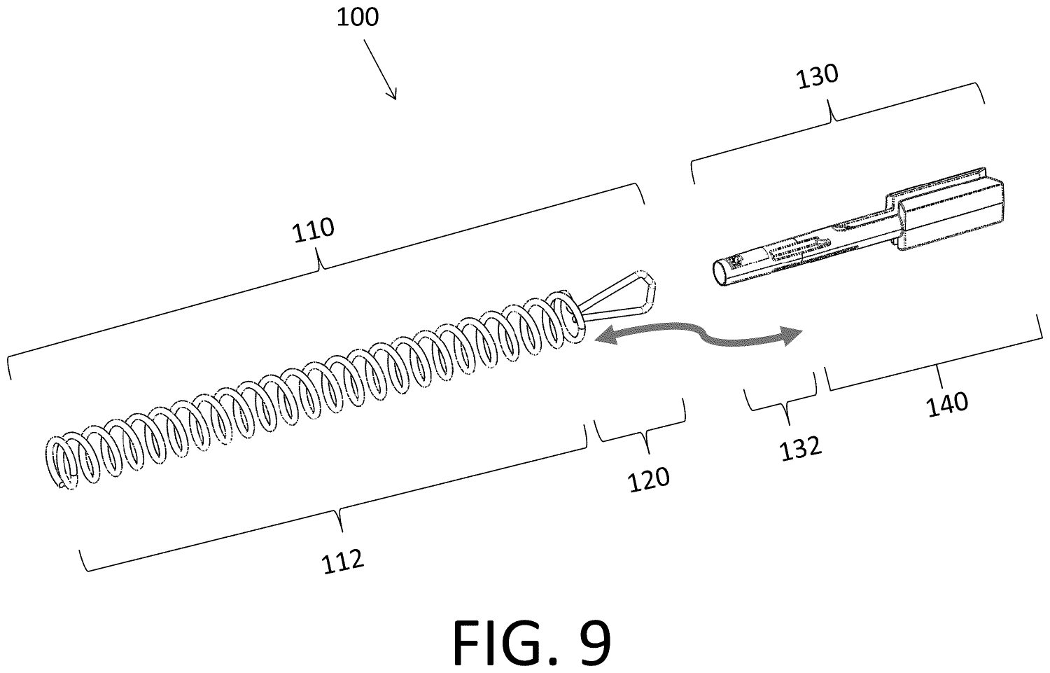

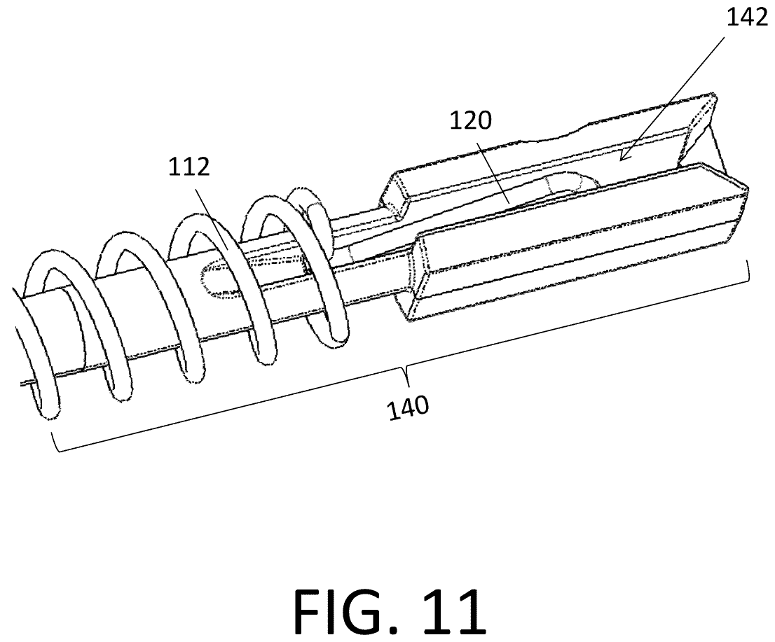

FIG. 9 is a detailed perspective view illustrating assembly of the spring/latch 110 and the needle catch/trigger 130 during assembly of the plunger member 70 (see FIG. 7). The spring/latch 110 and the needle catch/trigger 130 are aligned along a longitudinal axis, then brought toward each other such that the latch portion 120 of the spring/latch 110 is disposed in the slot 142 formed in the trigger portion 140 of the needle catch/trigger 130 as shown in FIGS. 10 and 11.

FIG. 10 is a mixed perspective (of spring/latch 110) and cross-sectional (of needle catch/trigger 130) view showing assembly of the needle retraction system 100 (i.e., the spring/latch 110 and the needle catch/trigger 130). When assembled as shown in FIG. 10, a proximal end 122 of the latch portion 120 of the spring/latch 110 is in contact with and inclined surface/portion 144 of the trigger portion 140 of the needle catch/trigger 130. Interactions between the latch portion 120 and the trigger portion 140 to latch and unlatched the spring portion 112 are further described herein.

FIG. 11 is a detailed top perspective view of the spring/latch 110 and the needle catch/trigger 130 after assembly. The latch portion 120 of the spring/latch 110 is disposed in the slot 142 formed in the trigger portion 140 of the needle catch/trigger 130. The wire loops of the spring portion 112 of the spring/latch 110 are wrapped around the needle catch portion 132 of the needle catch/trigger 130. During assembly, the latch portion 120 may be tilted upward to allow the latch portion 120 to slide into the slot 142.

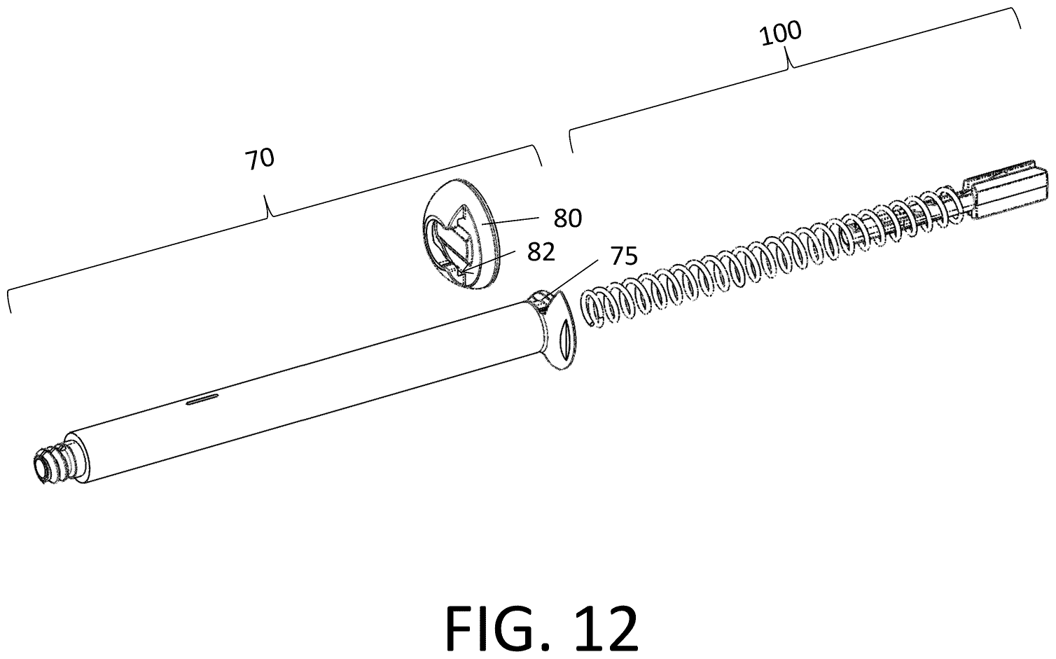

FIG. 12 is a perspective view showing the next step in assembly of the plunger member 70. After the spring/latch 110 and the needle catch/trigger 130 are assembled as shown in FIG. 10, the needle retraction system 100 can be inserted into the plunger interior 74 through a proximal opening 76 in the plunger housing 72.

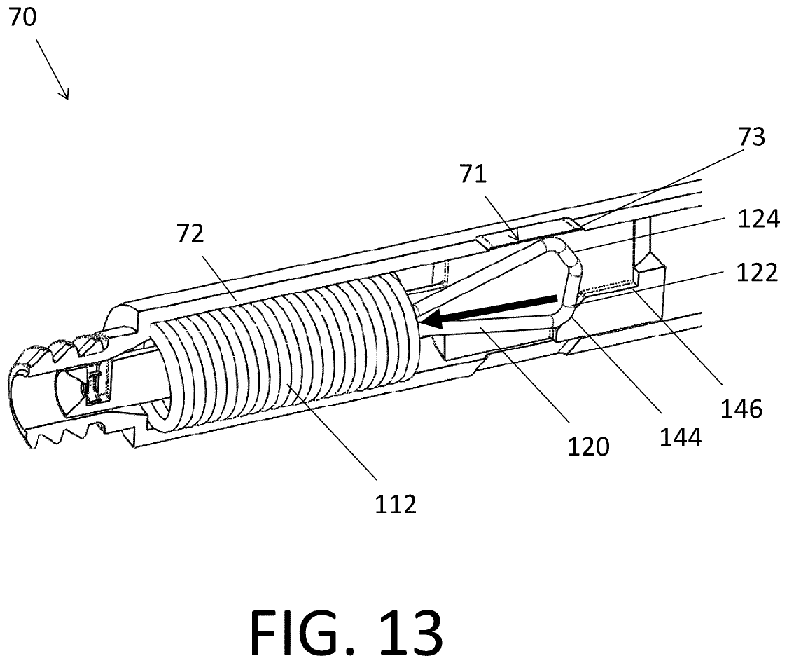

FIG. 13 shows the spring portion 112 of the spring/latch 110 compressed between a distal end 78 of the plunger housing 72 and a fixture (not pictured). This fixture can fit inside the plunger interior 74 and can bypass the trigger so that the latch hook remains in the relaxed configuration. The latch portion 120 of the spring/latch 110 is urged in a distal direction by the inclined surface 144 of the trigger portion 140 of the needle catch/trigger 130 as the needle catch/trigger 130 is inserted distally into the plunger interior 74. In FIG. 13, the latch portion 120 of the spring/latch 110 is still in a relaxed configuration (i.e., completely inside of the plunger housing 72).

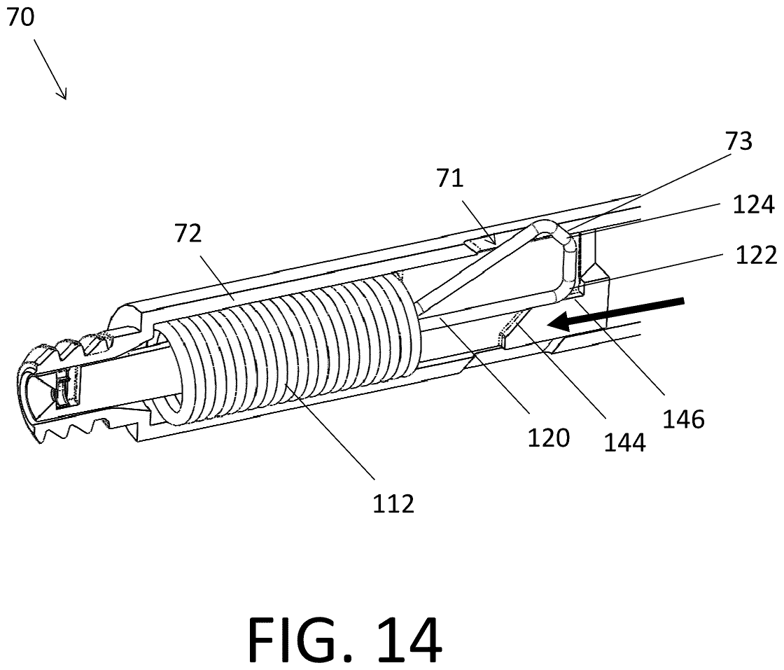

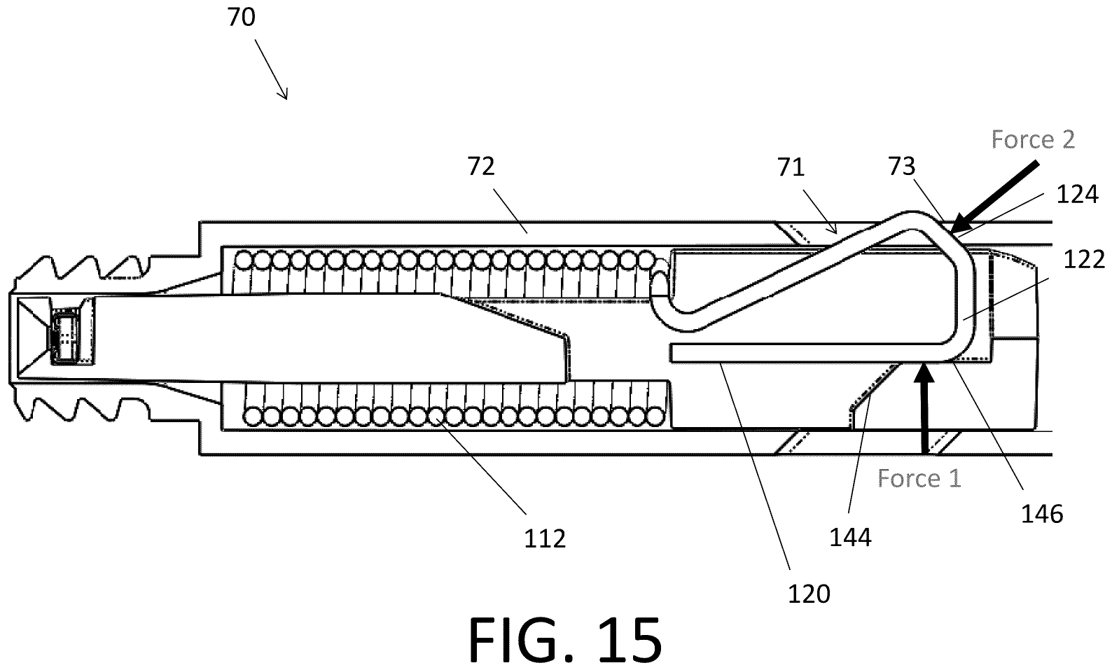

FIGS. 14 and 15 show the latch portion 120 of the spring/latch 110 after it has been moved partially out of the plunger interior 74 by continued distal insertion of the needle catch/trigger 130 into an extended/latched configuration. In particular, the inclined surface 144 of the trigger portion 140 of the needle catch/trigger 130 redirects distally directed force orthogonally to move the proximal end 122 of the latch portion 120 radially outward/upward and out of the plunger interior 74 through the lateral opening 71 defined by the plunger housing 72. Continued distal movement of the trigger portion 140 relative to the latch portion 120 places the latch portion 120 on top of a longitudinal surface 146 of the trigger portion 140. The longitudinal surface 146 exerts a resistive force (see Force 1 in FIG. 15) against the bias of the latch portion 120 to rotate out of the latched configuration. This contact also creates a frictional force between the proximal end 122 of the latch portion 120, and the longitudinal surface 146, which acts orthogonally to Force 1 (see FIG. 14). This frictional force helps to pin the trigger portion 140 in place in the plunger interior 74. While the longitudinal surface 146 depicted in FIGS. 13 to 15 is parallel to a longitudinal axis of the plunger member 70, in other embodiments, the surface can also be biased or angled in such a way to oppose the proximal movement of the trigger portion 140 with respect to the proximal end 122 of the latch portion 120, and hence oppose rotation of the latch portion 120 out of the latched configuration. For instance, by having a surface that is angled downward in a proximal direction, the downward bias of the proximal end 122 of the latch portion 120 can resist proximal movement of the trigger portion 140 with respect to the proximal end 122 of the latch portion 120.

Moving the proximal end 122 of the latch portion 120 out through the lateral opening 71 juxtaposes a latching surface 124 of the latch portion 120 adjacent a proximal wall 73 defining the lateral opening 71. The proximal wall 73 exerts a compression force (see Force 2 in FIG. 15) against the latching surface 124, which cooperates with Force 1 to retain the latch portion 120 in the latched configuration depicted in FIGS. 14 and 15. The latching surface 124 of the latch portion 120 is rigidly connected to the coiled spring portion 112 via an almost vertically oriented wire which ends nearly at the center of the end coil of the spring. Therefore, the compression force exerted by the proximal wall 73 acts through this vertically oriented wire to keep the coiled spring portion 112 flatly compressed.

When the latch portion 120 is in the latched configuration, the spring portion 112 of the spring/latch 110 is compressed between a distal end 78 of the plunger housing 72 and the proximal wall 73 defining the lateral opening 71 with the compression force (Force 2) being transmitted through the latch portion 120 to the spring portion 112 of the spring/latch 110. As long as the latch portion 120 is in the latched configuration, the spring portion 112 of the spring/latch 110 is held in a compressed configuration shown FIGS. 13 and 14. In the compressed configuration, the spring portion 112 is biased to expand into the relaxed configuration shown in FIG. 11.

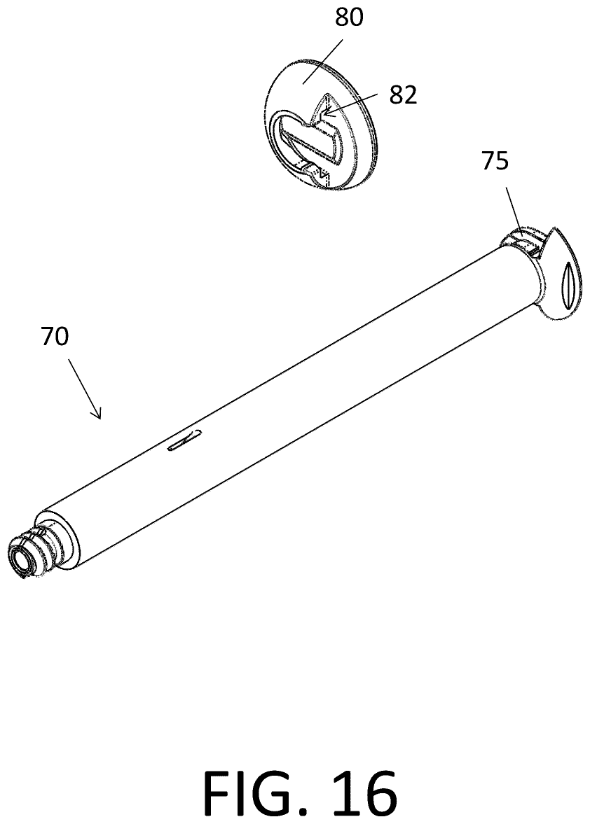

FIG. 16 depicts the next step in assembly of the plunger member 70 after the spring/latch 110 that and the needle catch/trigger 130 is assembled and inserted into the plunger interior 74, the latch portion 120 has been placed in the latched configuration, and the spring portion 112 has been placed in the compressed configuration. At this point, the proximal end cap 80 is ready to be slid onto the plunger body 72 to close the proximal opening 76 therein.

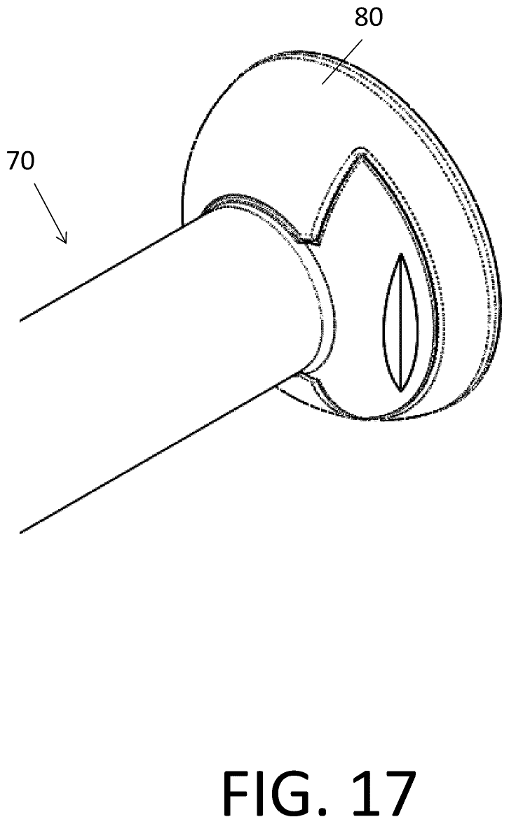

FIG. 17 shows the plunger member 70 after the proximal end cap 80 has been slid onto the plunger body 72 to close the proximal opening 76 therein. As shown in FIG. 16, the proximal end cap 80 includes a slot 82 configured to receive and interfere with a tongue 75 formed at the proximal end of the plunger body 72 to secure the proximal end cap 80 on to the plunger body 72. Snapping on the proximal end cap 80 eliminates the need for ultrasonic welding and inadvertent welding of plunger member 70 and needle retraction system 100 components. The tongue 75 acts like a springy dive board within slot 82, which can dampen/minimize the impact from the spring portion 112 to a user's body. The slot 82 and the tongue 75 may also be configured such that a cushion 84 (e.g., a space) is formed in the proximal end cap 80 when assembled with the plunger body 72. The cushion 84 may be configured to minimize transmission of force from the spring portion 110 to a user's body (e.g., thumb) during needle retraction. The cushion 84 may also be configured to minimize sound generated during needle retraction. While the embodiment depicted in FIGS. 16 and 17 include both a tongue 75 and a cushion 84, other embodiments may include only one of these two components, which may act independently to dampen/minimize the impact from the spring portion 112 to a user's body.

### Exemplary Safe Injection Systems and Methods

FIGS. 18 to 27 depict use of a safe injection system 50 including a plunger member 70 with a needle retraction system 100 to perform a safe injection according to some embodiments.

FIG. 18 is a side cross-sectional view depicting an assembled safe injection system 50 in a ready to use configuration including the plunger member 70 depicted in FIGS. 7 to 17 according to some embodiments. The plunger member 70 with the latch portion 120 in the extended/latched configuration is coupled to a stopper member 36 (e.g., using a threaded connection). Then the stopper member 36 along with the plunger member 70 attached thereto are inserted into the syringe body 34. In some embodiments, the syringe body 34 may be prefilled with an injectable liquid. In alternative embodiments, the stopper member 36 may first be inserted into the syringe body 34, and then the plunger member 70 may be coupled to the stopper member 36.

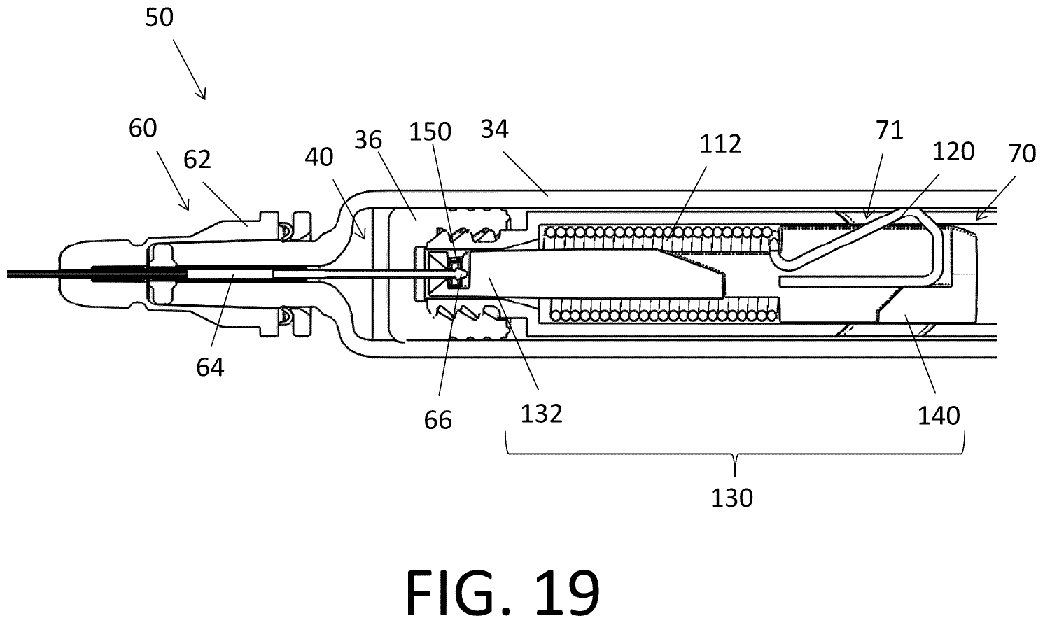

FIG. 19 is a side cross-sectional view depicting the next step in safe injection of an injectable liquid using a safe injection system 50 according to some embodiments. The application of distally directed force (e.g., by a user's thumb) against the proximal end cap 80 drives the plunger member 70 and the stopper member 36 attached thereto distally within the syringe body 34 to eject the injectable fluid contained therein out of the needle 64 to complete the injection. During injection, the latch portion 120 of the spring/latch 110 is retained in the extended/latched configuration shown in FIG. 19 by the longitudinal surface 146 of the trigger portion 140 of the needle catch/trigger 130.

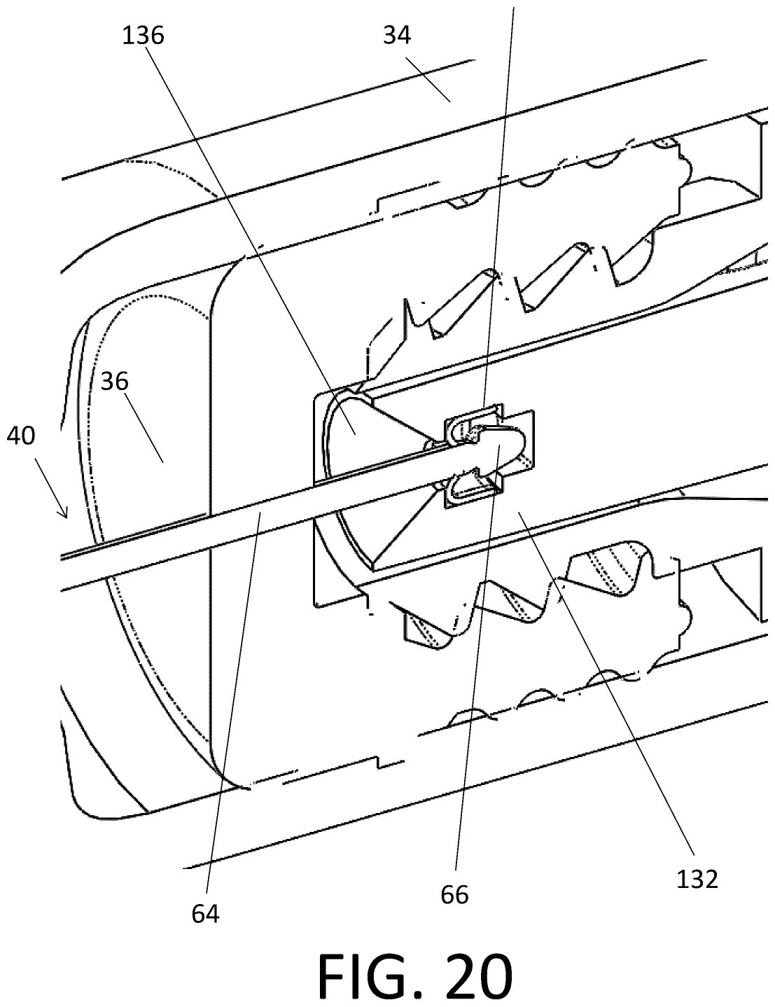

FIG. 20 is a detailed perspective cross-sectional view showing a proximal end geometric feature 66 of a needle 64 captured by the needle catch portion 132 of the needle catch/trigger 130 according to some embodiments. The needle catch portion 132 includes a funnel 136 and a needle catch disc 150 disposed in a space 134 defined by the needle catch portion 132. The proximal end geometric feature 66 includes an enlarged proximal end configured to pass through the needle catch disc 150 in the proximal direction but to interfere with the needle catch disc 150 in the distal direction. The needle catch disc 150 may be pre-bent to reduce the force needed to move the proximal end geometric feature 66 proximally past the needle catch disc 150. In some embodiments, the amount of force needed to move the proximal end geometric feature 66 proximally past the needle catch disc 150 is less than 2 pounds such that the capture of the proximal end geometric feature 66 in the needle catch disc 150 is not noticeable to many users.

The proximal end geometric feature 66 may be stamped or cold-formed into a sharp arrow shape to allow for lower insertion force needed to move the proximal end geometric feature 66 proximally past the needle catch disc 150. The needle catch disc 150 may be cup shaped to provide higher retention strength due to greater elastic properties. Both of these features allow for lower insertion force needed to move the proximal end geometric feature 66 proximally past the needle catch disc 150.

Further, the needle catch/trigger 130 and the plunger housing 72 may be configured such that the frictional force between these two components is approximately 2 to 5 pounds. Accordingly, the proximal end geometric feature 66 may be moved into the needle catch disc 150 by distally directed force through the plunger member 70 and the stopper member 36 without moving the needle catch/trigger 130 proximally relative to the plunger housing 72.

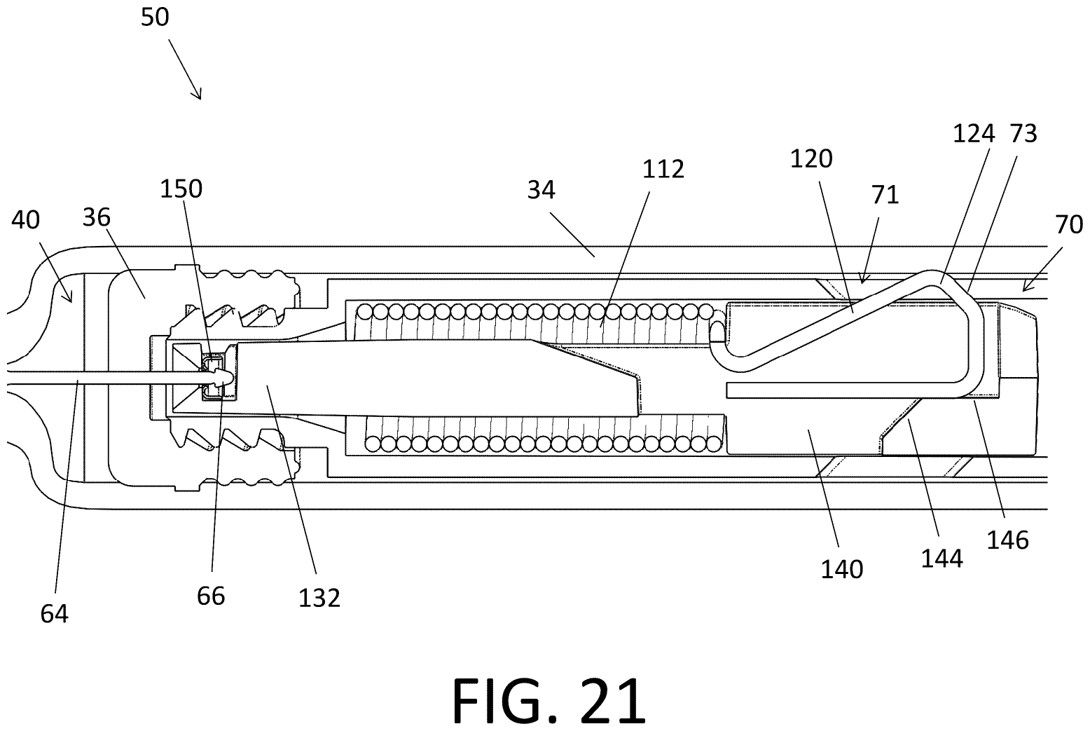

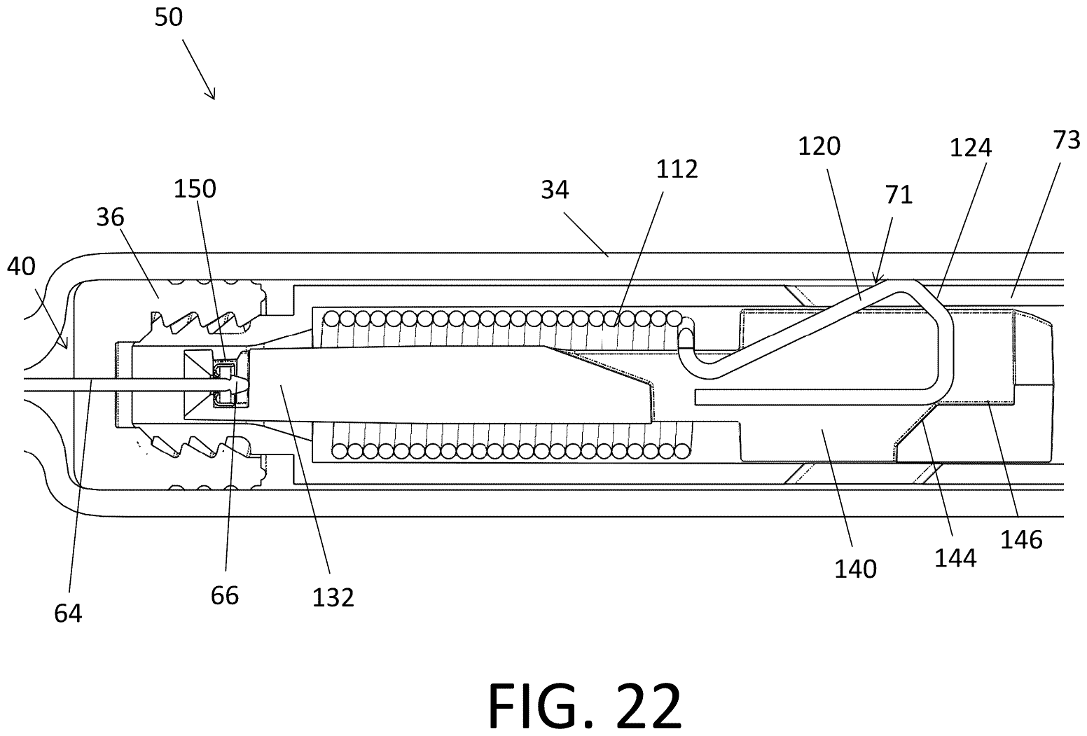

FIGS. 21 and 22 are side cross-sectional views that illustrate unlatching of the latch portion 120 of the spring/latch 110 and retraction of the needle 64 into the plunger housing 72 according to some embodiments. FIG. 21 shows the proximal end geometric feature 66 captured in the needle catch disc 150, thereby preventing distal movement of the proximal end geometric feature 66 relative to the needle catch disc 150. In FIG. 21, the longitudinal surface 146 of the trigger portion 140 of the needle catch/trigger 130 retains the latch portion 120 in the extended/latched configuration through the lateral opening 71 in the plunger housing 72.

With continued application of distally directed force through the plunger member 70, the proximal end geometric feature 66 of the needle 64 is moved proximally relative to the needle catch portion 132 (or the plunger member 70 is moved distally relative to the proximal end geometric feature 66) as shown in FIG. 22. Proximal movement pushes the proximal end geometric feature 66 against the proximal wall of the space 134 in the needle catch portion 132, thereby moving the needle catch/trigger 130 proximally relative to the spring/latch 110.

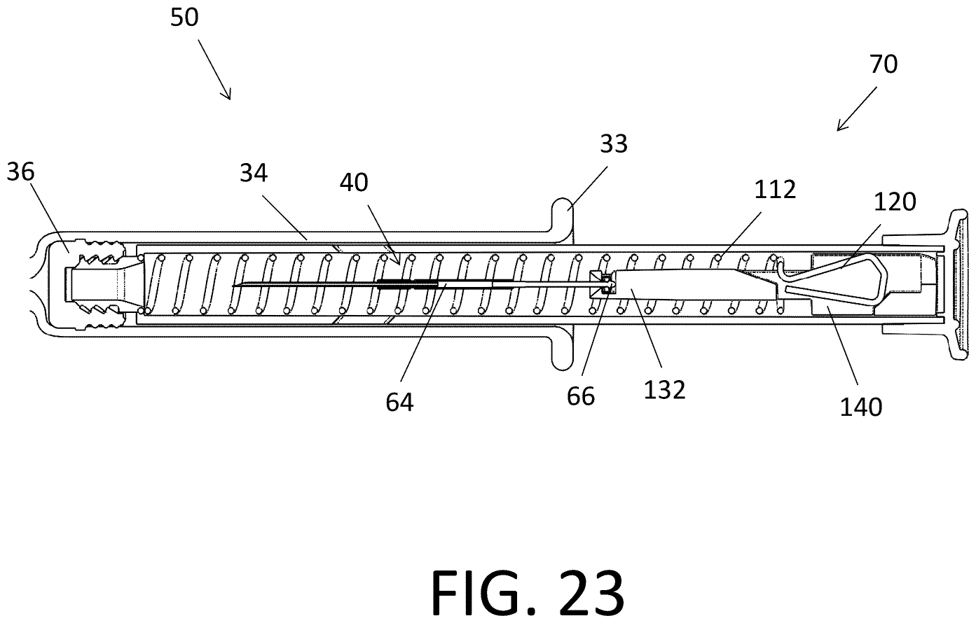

When the longitudinal surface 146 of the trigger portion 140 moves proximally past the latch portion 120, the latch portion 120 is no longer supported by the longitudinal surface 146 as shown in FIG. 23. With the resistive force previously provided by the longitudinal surface 146 removed, the bias/moment exerted on the latch portion 120 rotates the latch portion 120 away from the lateral opening 71 and back into the plunger interior 74 and into the relaxed configuration (e.g., FIG. 13). The moment exerted on latch portion 120 is mostly due to the compressed spring portion 112. This force, Force 2 (which is the only reaction force against the compression force from the spring portion 112; see FIG. 15), pivots the latch portion 120 about a pivot described by the curved portion of the connecting wire between the proximal end of the spring portion 112 and the latch portion 120. This moment pivots the latch portion 120 into the relaxed configuration after Force 1 (see FIG. 15) is removed by proximal movement of the trigger portion 140. Rotating the latch portion 120 into the relaxed configuration removes the resistive force provided by the proximal wall 73 defining the lateral opening 71 against proximal movement of the latching surface 124 of the latch portion 120 relative to the plunger housing 72. The distally directed slant of both the proximal wall 73 and the latching 124 facilitates unlatching of the latch portion 120. The proximal end 122 of the latch portion 120 provides strength for the latch portion 120 to keep the latch portion 120 in the latched configuration.

Without the resistive force previously provided by the proximal wall 73, the compressed spring portion 112 expands proximally and exerts a proximally directed force on the needle catch/trigger 130, thereby moving the needle catch/trigger 130 and the needle 64 attached thereto in a proximal direction into the plunger housing 72. FIG. 23 shows the results of retraction of the needle 64 completely through the stopper member 36 and into the plunger housing 72. After retraction of the needle 76 into the plunger housing 72, the safe injection system 50 is in a safe configuration wherein accidental needle sticks are prevented and the safe injection system 50 may be safely disposed of.

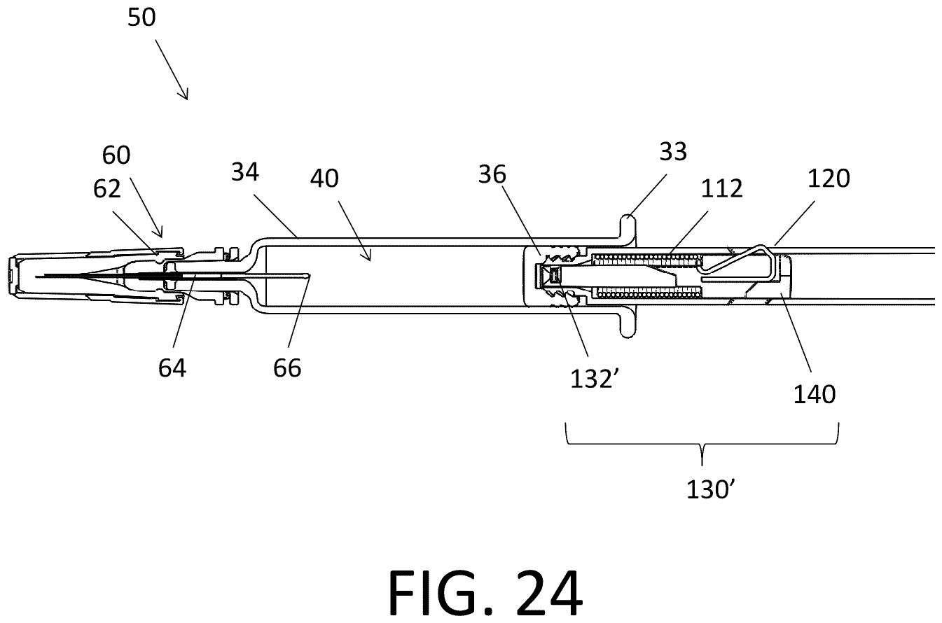

FIGS. 24 to 27 depict use of a safe injection system 50′ including a plunger member 70′ with a needle retraction system 100′ to perform a safe injection according to some embodiments.

FIG. 24 is a side cross-sectional view depicting an assembled safe injection system 50′ in a ready to use configuration including a plunger member 70′ according to some embodiments. The plunger member 70′ is similar to the plunger member 70 depicted in FIGS. 7 to 17. The difference between the plunger member 70′ depicted in FIGS. 24 to 27 and the plunger member 70 depicted in FIGS. 7 to 17 is found in the needle catch portion 132′ of the needle catch/trigger 130′. Instead of a needle catch disc 150 disposed in a space 134 defined by the needle catch portion 132 (see e.g., FIG. 20), the needle catch portion 132′ of the needle catch/trigger 130′ depicted in FIGS. 24 to 27 include a pair of self-energizing tabs 138 that form the funnel 136′ in the needle catch portion 132′. The pair of self-energizing tabs 138 may be formed by a slot cut through the funnel 136′. The needle catch portion 132′ also includes windows 139 to increase flexibility of the self-energizing tabs 138, thereby lowering insertion force needed to move the proximal end geometric feature 66 proximally past the pair of self-energizing tabs 138. The proximal ends of the self-energizing tabs 138 may have additional thickness to increase the strength of the proximal end geometric feature 66/needle catch portion 132′ connection during proximal retraction.

FIG. 24 depicts the plunger member 70′ with the latch portion 120′ in the extended/latched configuration and coupled to a stopper member 36 (e.g., using a threaded connection) in a syringe body 34. As such, the safe injection system 50′ depicted in FIG. 24 is in a ready to use configuration.

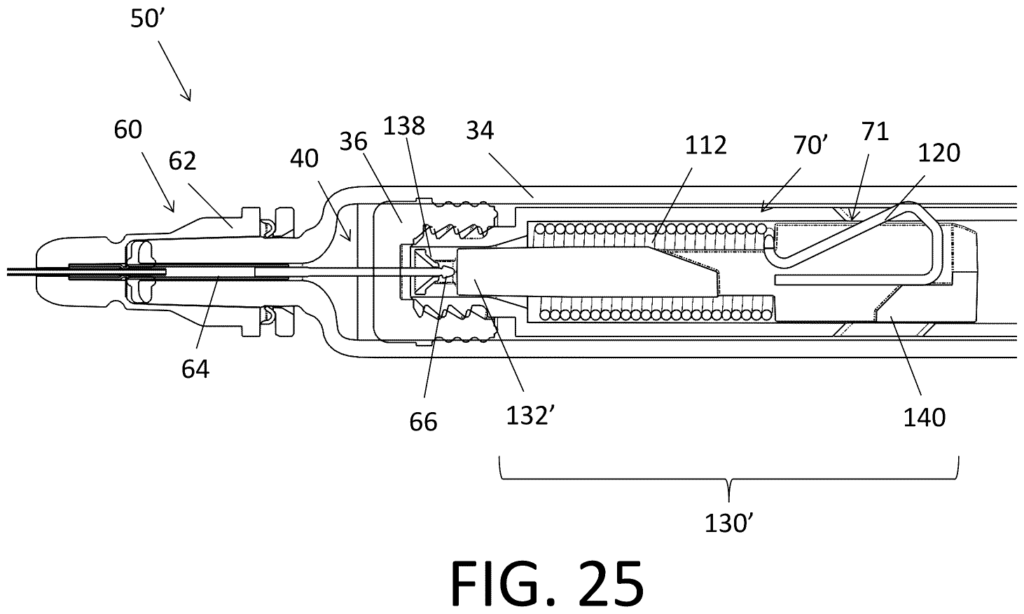

FIG. 25 is a side cross-sectional view depicting the next step in safe injection of an injectable liquid using a safe injection system 50′ according to some embodiments. The application of distally directed force (e.g., by a user's thumb) against the proximal end cap 80 drives the plunger member 70′ and the stopper member 36 attached thereto distally within the syringe body 34 to eject the injectable fluid contained therein out of the needle 64 to complete the injection. During injection, the latch portion 120 of the spring/latch 110 is retained in the extended/latched configuration shown in FIG. 24 by the longitudinal surface 146 of the trigger portion 140 of the needle catch/trigger 130.

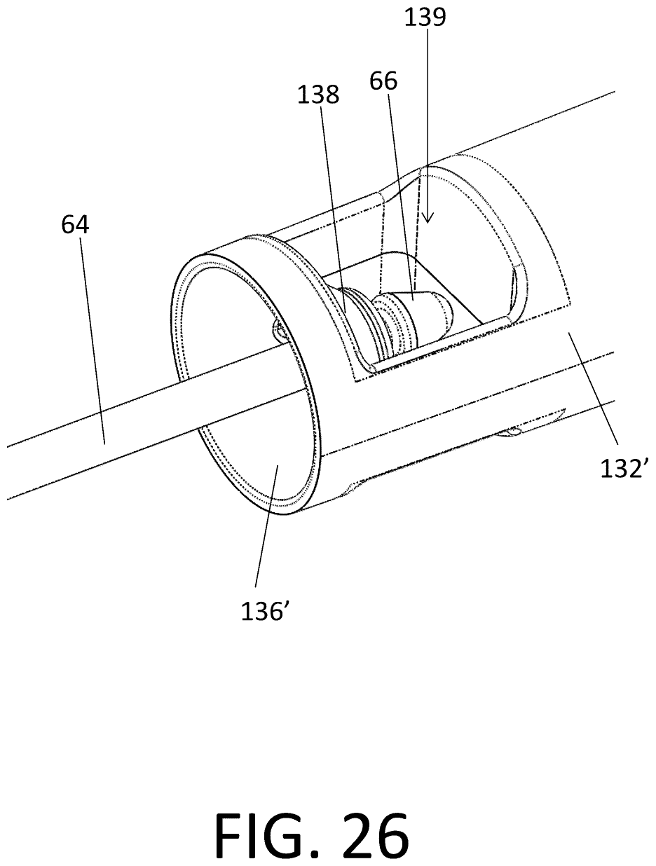

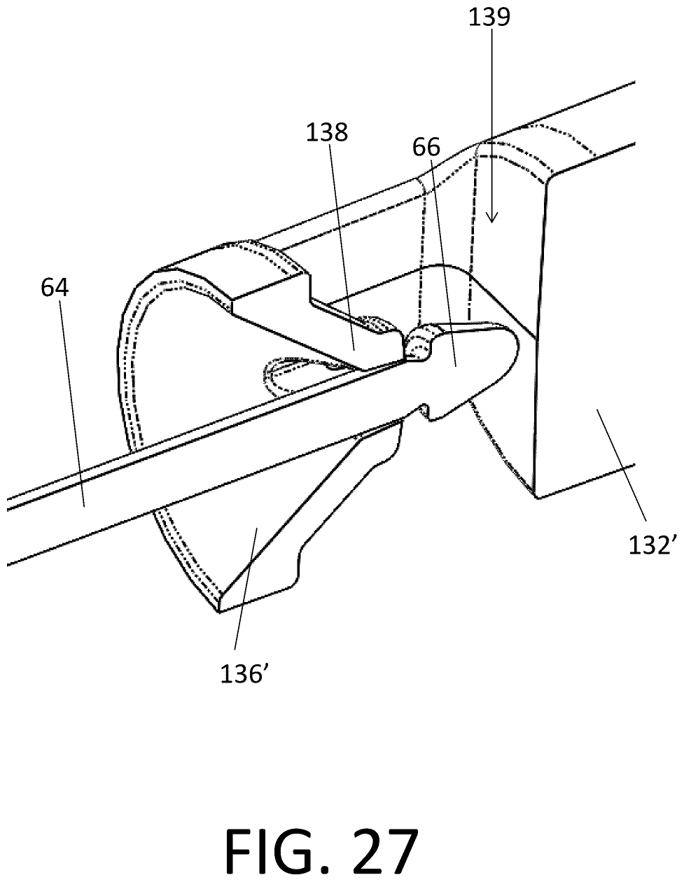

FIGS. 26 and 27 are detailed perspective (FIG. 26) and (FIG. 27) detailed perspective cross-sectional views showing a proximal end geometric feature 66 of a needle 64 captured by the needle catch portion 132′ of the needle catch/trigger 130′ according to some embodiments. As described above, the needle catch portion 132′ of the needle catch/trigger 130′ depicted in FIGS. 24 to 27 include a pair of self-energizing tabs 138 that form the funnel 136′ in the needle catch portion 132′. The proximal end geometric feature 66 includes an enlarged proximal end configured to pass through the self-energizing tabs 138 in the proximal direction but to interfere with the self-energizing tabs 138 in the distal direction. In some embodiments, the amount of force needed to move the proximal end geometric feature 66 proximally past the self-energizing tabs 138 is less than 2 pounds such that the capture of the proximal end geometric feature 66 in the self-energizing tabs 138 is not noticeable to many users.

Further, the needle catch/trigger 130′ and the plunger housing 72 may be configured such that the frictional force between these two components is approximately 2 to 5 pounds. Accordingly, the proximal end geometric feature 66 may be moved into the self-energizing tabs 138 by distally directed force through the plunger member 70′ and the stopper member 36 without moving the needle catch/trigger 130′ proximally relative to the plunger housing 72.

After the proximal end geometric feature 66 of a needle 64 is captured by the needle catch portion 132′ of the needle catch/trigger 130′ (i.e., the self-energizing tabs 138 thereof), the latch portion 120 may be moved from the extended/latch configuration to a relax configuration and the needle 76 may be retracted by the spring portion 112 into the plunger housing 72 as shown in FIGS. 21 to 23 and described above.

The needle retraction systems 100, 100′ described herein have fewer components while accurately and precisely withdrawing needles 76 at least partially into injection systems 50, 50′ after completing injections to provide safe injection systems that minimize accidental needle sticks. The safe injection systems 50, 50′ may also utilize the existing and relatively well-controlled supply chain of conventionally delivered off-the-shelf components, and the corresponding assembly machinery and personnel.

While various embodiments have been described with specific connectors (e.g., slip and Luer), these embodiments can be used with any known injection system connectors. While various embodiments have been described with staked needles and needle connectors, these embodiments can be used with any known permanently coupled needle or needle connector system.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. A system for safe injections, comprising:
a syringe body having proximal and distal ends, a syringe interior, and a syringe flange at the proximal end thereof;
a needle hub assembly coupled to the distal end of the syringe body, wherein the needle hub assembly comprises:
a needle hub coupled to the distal end of the syringe body; and
a needle removably coupled to the needle hub;
a stopper member disposed in the syringe interior; and
a plunger member coupled to the stopper member, wherein the plunger member comprises:
a plunger housing defining a plunger interior;
a spring and a latch, wherein the spring and the latch are integrally formed; and
a needle catch and a trigger, wherein the needle catch and the trigger are integrally formed.

2. The system of claim 1, wherein the spring is disposed in the plunger interior.

3. The system of claim 1, wherein the needle catch is disposed in the plunger interior.

4. The system of claim 1, wherein the trigger is disposed in the plunger interior.

5. The system of claim 1, wherein the latch is disposed at least partially in the plunger interior,
wherein the plunger body further defines a lateral opening therein,
wherein the latch has an extended configuration and a relaxed configuration,
wherein when the latch is in the extended configuration, the latch is configured to extend out of the plunger interior through the lateral opening and to interfere with the plunger body adjacent the lateral opening to limit proximal movement of the latch relative to the plunger body, and
wherein when the latch is in the extended configuration, the latch compresses the spring.

6. The system of claim 1, wherein the spring comprises a stack of loops formed from a wire,
wherein the latch comprises an orthogonal loop formed from the wire and orthogonal to the stack of loops comprising the spring.

7. The system of claim 6, wherein the orthogonal loop comprises an opening smaller than a width of the wire.

8. The system of claim 6, wherein the orthogonal loop comprises a straight section.

9. The system of claim 1, wherein the needle comprises a geometric feature at a proximal end thereof,
wherein the needle catch defines
a funnel at a distal end thereof, and
an opening in the center of the funnel,
wherein the funnel is configured to guide the geometric feature of the needle into the opening and the needle catch,
wherein the needle catch comprises a needle latch configured to limit distal movement of the geometric feature of the needle relative to the funnel while allowing proximal movement of the geometric feature of the needle relative to the funnel.

10. The system of claim 9, wherein the needle latch is self-energizing.

11. The system of claim 9, wherein the needle latch comprises a needle catch disc disposed in a space defined by the needle catch proximal of the funnel.

12. The system of claim 11, wherein the needle catch disc comprises first and second tabs.

13. The system of claim 12, wherein the first and second tabs are pre-bent in a proximal direction.

14. The system of claim 12, wherein the first tab is shorter than the second tab,
wherein the first tab is configured to not bend when the geometric feature of the needle moves proximally past the needle catch disc, and
wherein the second tab is configured to at least partially elastically bend in a proximal direction to allow the geometric feature of the needle to move proximally past the needle catch disc.

15. The system of claim 12, wherein the first and second tabs are each configured to at least partially elastically bend in a proximal direction to allow the geometric feature of the needle to move proximally past the needle catch disc.

16. The system of claim 1, wherein the trigger defines a slot configured to receive the latch.

17. The system of claim 1, wherein the trigger defines an inclined surface configured to move the latch from a relaxed configuration to an extended configuration when the latch moves proximally over the included surface, and
wherein the trigger and the catch are configured such that moving the plunger member to position the stopper member at the distal end of the syringe body causes the needle to
at partially penetrate the stopper member,
engage with the needle catch, and
push the trigger proximally relative to the plunger housing and the latch to move the inclined surface proximally away from the latch, and to allow the latch to move from the extended configuration to the relaxed configuration.

18. The system of claim 1, wherein the plunger member comprises a proximal end cap slidably coupled to a proximal end of the plunger housing.

19. The system of claim 18, wherein the proximal end cap defines a space configured to minimize transmission of force from the spring upon retraction of the needle at least partially into the plunger interior.

20. The system of claim 18, wherein the proximal end cap defines a space configured to minimize a sound resulting from retraction of the needle at least partially into the plunger interior.
